(19) United States
(12) Patent Application Publication
(10) Pub. No.: US 2018/0049672 A1
(43) Pub. Date: Feb. 22, 2018

(54) INDEXING TAPE FOR DETERMINING VENTILATOR SETTINGS

(71) Applicant: Valerie REID, Douglasville, GA (US)

(72) Inventor: Valerie REID, Douglasville, GA (US)

(73) Assignee: "REID" RAPID REFERENCES, LLC, Douglasville, GA (US)

(21) Appl. No.: 15/599,335

(22) Filed: May 18, 2017

Related U.S. Application Data
(60) Provisional application No. 62/375,517, filed on Aug. 16, 2016.

(51) Int. Cl. A61B 5/107; A61M 16/06; G01B 3/00; G01B 3/10

(52) U.S. Cl. CPC A61B 5/1072; A61M 16/06; A61M 2230/40; G01B 3/1082; G01B 3/1084; G01B 3/004

(57) ABSTRACT

An indexing tape for determining respiratory tidal volume settings of a ventilator for a human patient. The tape includes an elongate body, a reference element of the tape body configured for positioning at a first end of the patient, a series of length-indicating elements of the tape body spaced from the reference element and configured so that an aligned one at a second end of the patient indicates the patient's height, and a plurality of ventilator-setting elements of the tape body correlated to the length-indicating elements and including the tidal volumes. A method of determining ventilator settings includes positioning the tape along the patient with the reference element adjacent the patient first end and with one of the length-indicating elements adjacent and aligned with the patient second end, and reading the respective ventilator-setting element corresponding to the aligned length-indicating element to identify an appropriate tidal-volume setting for the ventilator.

### CROSS-REFERENCE TO RELATED APPLICATION

This application claims the priority benefit of U.S. Provisional Patent Application Ser. No. 62/375,517, filed Aug. 16, 2016, which is hereby incorporated herein by reference.

### TECHNICAL FIELD

The present invention relates generally to medical care for acutely injured and/or ill persons, and particularly to determining and providing proper ventilation for such critical-care patients.

### BACKGROUND

In the critical-care environment, minutes count when caring for an acutely injured or ill patient, for example during transport in an ambulance or medevac. For such acutely injured/ill patients, providing and maintaining the patient's needed oxygen inhalation and carbon dioxide exhalation is critical. So the vast majority of these critical-care patients are intubated with an endotracheal tube connected to a mechanical ventilator to provide them their needed breathing ventilation. Critical-care providers (e.g., paramedics, nurses, doctors, respiratory therapists, and other clinicians) are responsible for providing and maintaining the patient's needed respiration and ventilation through settings on the ventilator. Current best practices in medicine call for the critical-care provider to estimate the patient's height, calculate or look up the corresponding ideal weight for that height, and then calculate or look up the corresponding ventilator setting for that weight. But sometimes the critical-care provider overestimates or underestimates the patient's height, which results in an improper tidal volume provided to the patient.

Accordingly, it can be seen that there exists a need for a better way to quickly, easily, and accurately determine settings for ventilators for critical-care patients. It is to the provision of solutions to this and other problems that the present invention is primarily directed.

### SUMMARY

Generally described, the present invention relates to an indexing tape and method of use for determining respiration tidal volume settings of a ventilator for a human patient. The tape includes an elongate body, a reference element of the tape body configured for positioning at a first end (e.g., foot) of the patient, a series of length-indicating elements of the tape body spaced from the reference element and configured so that an aligned one at a second end (e.g., head) of the patient indicates the patient's approximate height, and a plurality of ventilator-setting elements of the tape body correlated to the length-indicating elements and including tidal volume settings for the ventilator. The method of determining ventilator settings includes positioning the tape along the patient with the reference element adjacent the patient first end (e.g., foot) and with one of the length-indicating elements adjacent and aligned with the patient second end (e.g., head), and reading the respective ventilator-setting element corresponding to the aligned length-indicating element to identify an appropriate tidal-volume setting for the ventilator.

The specific techniques and structures employed to improve over the drawbacks of the prior devices and accomplish the advantages described herein will become apparent from the following detailed description of example embodiments and the appended drawings and claims.

### BRIEF DESCRIPTION OF THE DRAWINGS

FIG. 1 is a side view of an indexing tape for determining ventilator settings according to an example embodiment of the present invention, showing the indexing tape in use with a patient.

FIG. 2 is a top view of the indexing tape and patient of FIG. 1.

FIG. 3 shows a detail portion the indexing tape and patient of FIG. 1 with a flap segment of the tape folded up and secured to the patient to prevent tape slippage.

FIG. 3A shows a detail portion of an alternative indexing tape and patient.

FIG. 4 is an end view of the indexing tape and patient portion of FIG. 3.

FIG. 5 shows a flap segment of the indexing tape of FIG. 3.

FIGS. 6-12 show a series of extension segments of the indexing tape of FIG. 3 that are arranged end-to-end and sequentially according to the depicted section lines A through G and that extend distally from the flap segment.

FIGS. 13-19 show a series of length-indicating segments of the indexing tape of FIG. 3 that are arranged end-to-end and sequentially according to the depicted section lines H through N and that extend distally from the last extension segment.

### DESCRIPTION OF EXAMPLE EMBODIMENTS

The present invention relates to an indexing tape for quickly and easily determining an appropriate respiration tidal-volume setting for a mechanical ventilator for patient. The indexing tape can be used with ventilators of a conventional type that can be connected to tubes and used for providing oxygen inhalation and carbon dioxide exhalation to patients in critical-care and other healthcare situations. Aspects of the invention include various embodiments of indexing tapes as well as methods of using the tapes for determining ventilator settings.

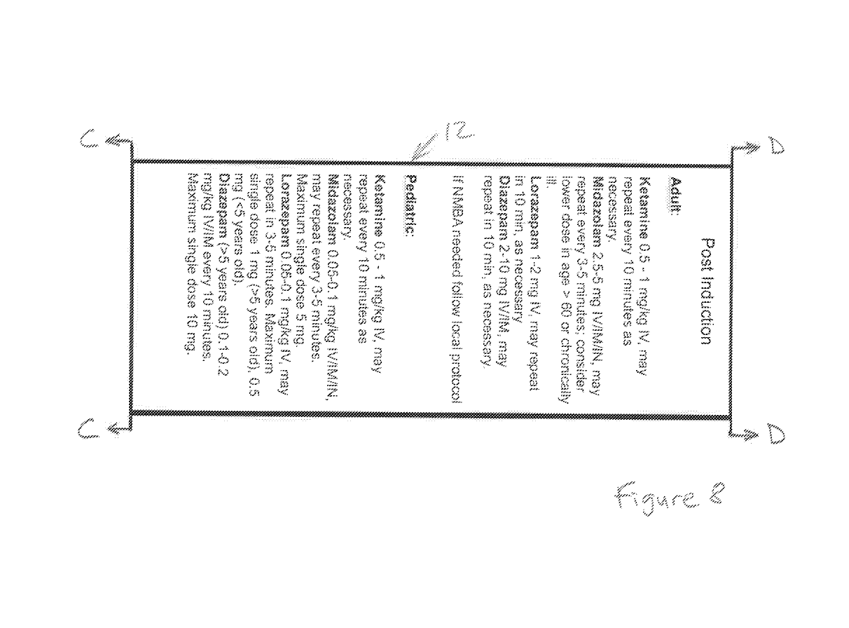

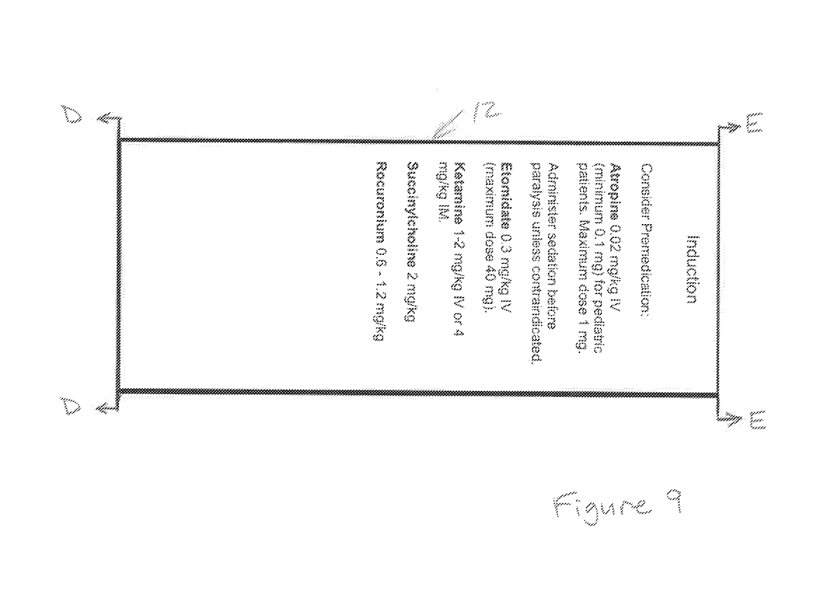

FIGS. 1-19 show an indexing tape 10, for determining respiration settings for ventilators (not shown) connected to tubes (not shown), according to an example embodiment of the invention. FIGS. 1-4 show examples of the tape 10 in use with a patient and FIGS. 5-19 show example segments of the continuous tape 10 (broken out into fifteen segments that align together as indicated by the matching sectioning lines).

The tape 10 includes an elongate body 12 with a length (extending between first and second ends 13 and 15 of the tape body) selected for use to measure adult humans and with a width (along all or at least portions of the length) selected for providing a display area for markings or other indicia. The tape body 12 is typically in the form of a conventional piece of ribbon or narrow strip of material, that is, relatively long and flat. And the tape body 12 has a width selected for displaying markings that are large enough to be legible and easily read by the naked human eye in normal daytime or illuminated-indoor lighting conditions (e.g., in an ambulance with the interior lights on). In the depicted embodiment, for example, the tape body 12 is about 90 inches long and about 2½ inches wide, with a thickness and flexibility providing for easily collapsing (e.g., rolling or folding) it into a compact configuration for storage and transport. The tape body 12 can be made for example of a sheet of a plastic, fabric, metal, vinyl, or other material selected to provide the desired flexibility and durability. In other embodiments, the tape body has other dimensions (e.g., longer or shorter, wider or narrower), has another shape (e.g., an oval or otherwise curved cross-section), and/or has other features for permitting collapsing into a compact configuration (e.g., a folding design with rigid segments hinged together, or a spring-biased automatic retracting design).

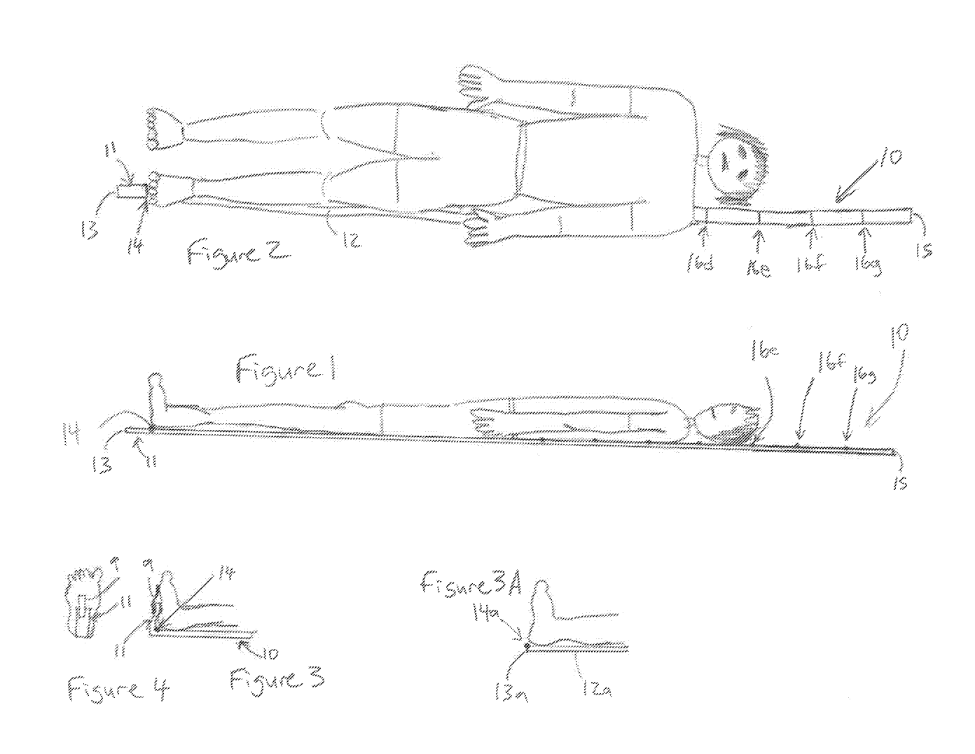

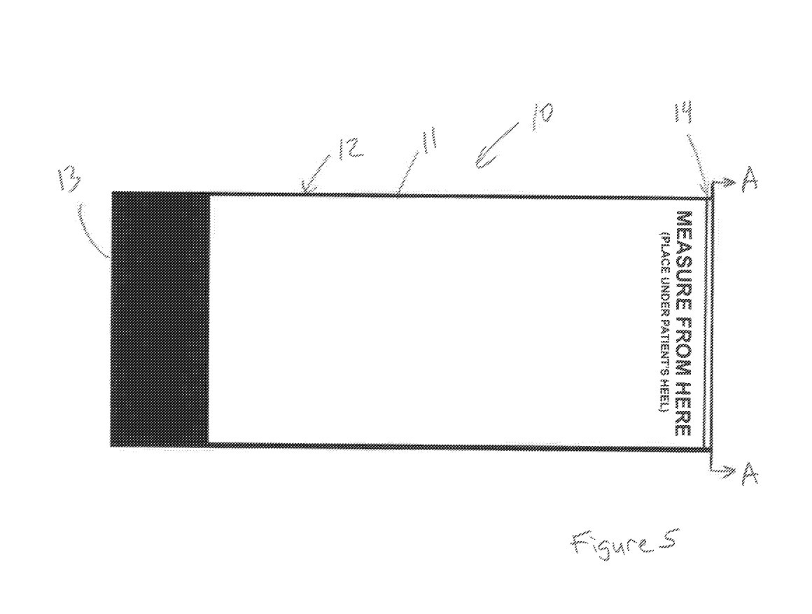

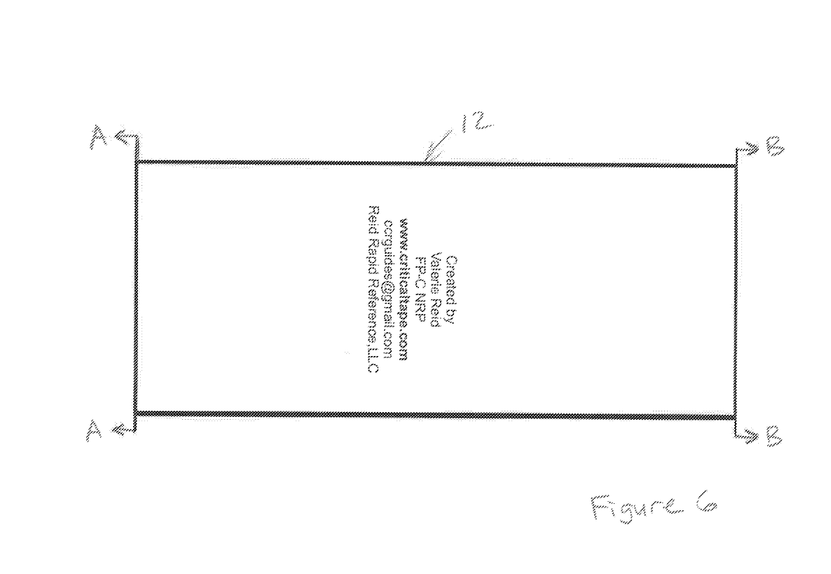

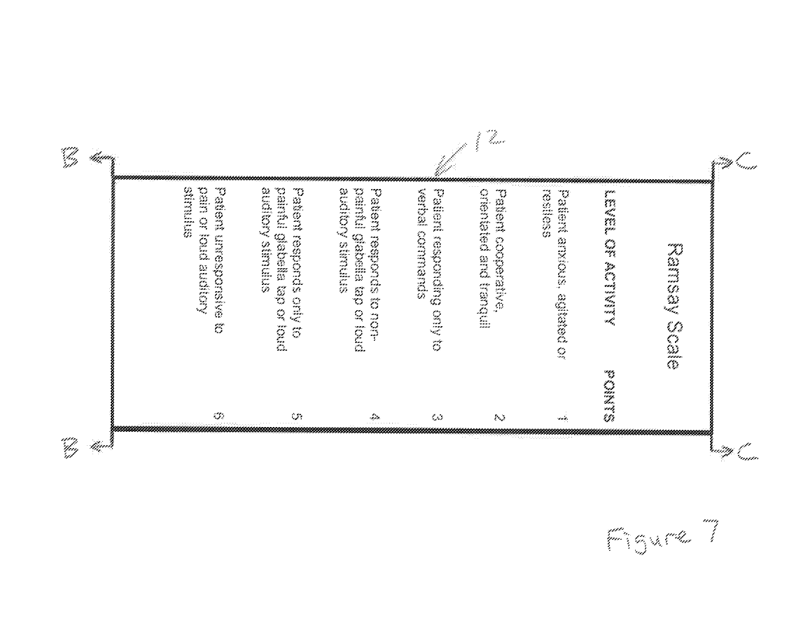

The tape body 12 includes a reference element 14, a plurality of length-indicating elements 16 serially arranged and spaced at increasing distances from the reference element, and a plurality of ventilator-setting elements 18 correlated to the length-indicating elements. The reference element 14 can be located distally offset from the first end 13 the tape body 12, as depicted in FIGS. 1-3, or alternatively the reference element 14a can be located at the first end 13a of the tape body 12a, as depicted in FIG. 3A. When offset distally from the first end 13 of the tape body 12, the length of the tape body between the first end and the reference element 14 forms a flap segment 11 that can be folded up onto the sole of the patient's foot and secured there for example by adhesive tape 9, as show in FIGS. 3-4.

The reference element 14 can be formed by a marking on the tape body 12, as depicted in FIGS. 1-5. For example, the reference element 14 can be made by printing, embossing, etching, or other conventional marking techniques and can be in the form of a prominent line extending part or all of the way across the width of the tape body 12 (e.g., a solid line, a dotted line, or a pair of parallel such lines). In other embodiments, the reference element can be formed by a mechanical element such as a tab, boss, groove, notch, or other conventional tactile and/or visual indicia. And in still other embodiments, the reference element or tape end can include a mechanical feature (e.g., a transverse tab/hook or a loop) for engagement by a body part of the patient (e.g., the foot or head) to secure the tape 10 in place during use to prevent tape slippage and thus prevent a patient-height misreading. In use, with the patient lying down (e.g., generally horizontally) on a support surface (e.g., a floor or the ground), the tape 10 is positioned on the support surface and along the patient with the reference element 14 at (adjacent, e.g., directly under or laterally offset, and aligned with) the sole of the patient's foot (e.g., at the heel) or the top/crown of the patient's head, as further described below. As used herein, the term “foot” means the patient's bare foot but also includes socks and/or shoes if worn by the patient during use of the tape 10.

The length-indicating elements 16 can be formed by markings on the tape body 12, as depicted in FIGS. 13-19. For example, the length-indicating elements 16 can be made by printing, embossing, etching, or other conventional marking techniques and can be in the form of scale lines extending part or all of the way across the width of the tape body 12 (e.g., solid lines, dotted lines, or pairs of parallel such lines). In other embodiments, the length-indicating elements can be formed by mechanical elements such as tabs, bosses, grooves, notches, or other conventional tactile and/or visual indicia. The length-indicating elements 16 are positioned on the tape body 12 at incrementally longer distances from the reference element 14 and correlated to identify incremental distances/lengths from the reference element.

As such, with the reference element 14 in position at one end (e.g., sole of foot) of the patient and with the tape body 12 in its extended/elongated use configuration positioned alongside and/or underneath the patient, the other end (e.g., top of head) of the patient will generally align with one of the length-indicating elements 16. That aligned length-indicating element 16 thus correlates to the approximate height of the patient and can be quickly and easily identified and read by a critical-care provider to correctly and reliably determine the patient's approximate height in tense and pressure-packed emergency situations.

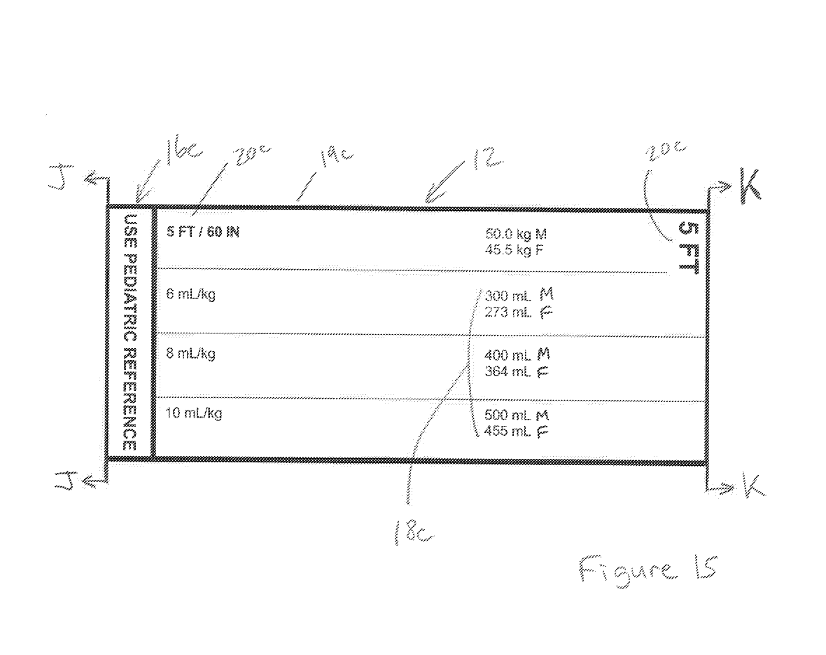

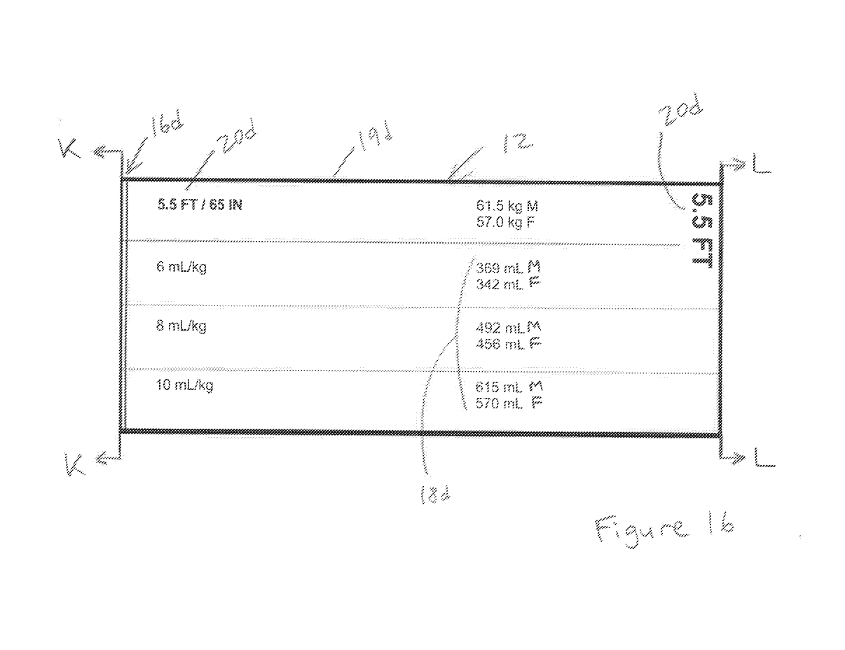

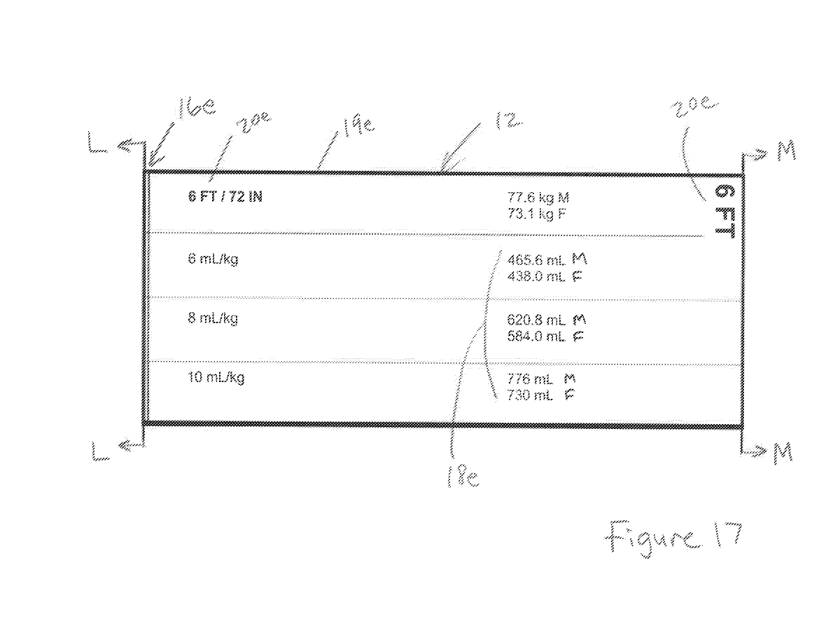

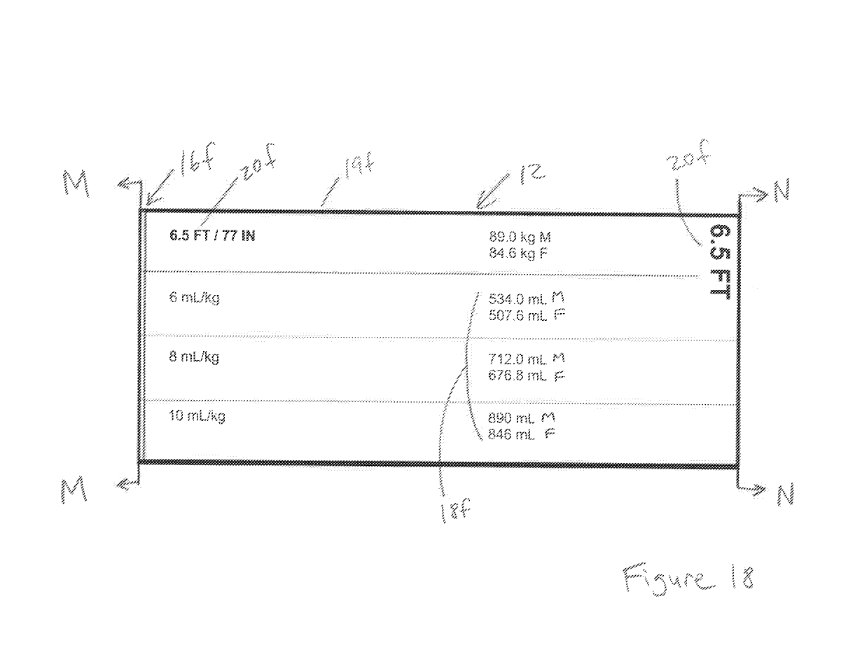

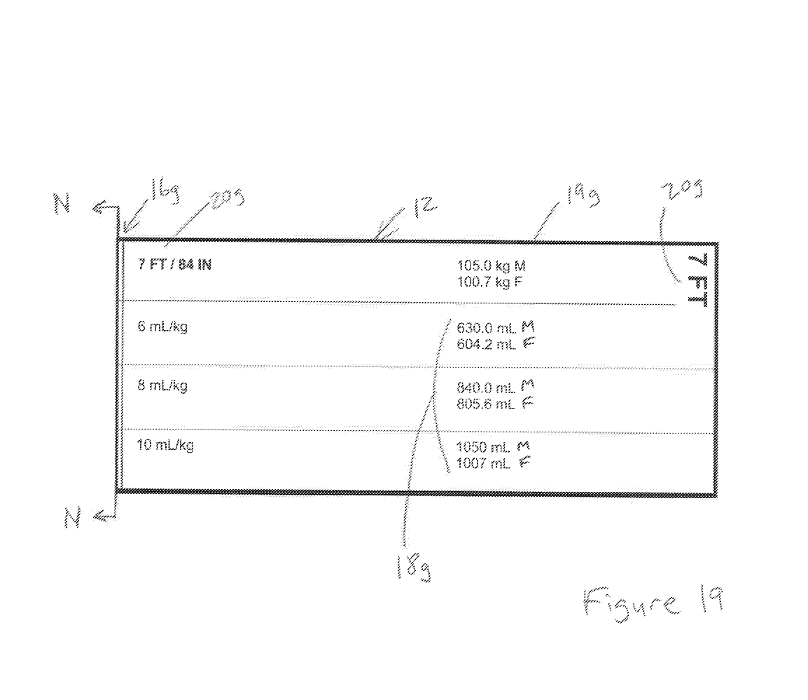

For example, the depicted embodiment includes a first length-indicating element 16a positioned at a first distance (4.0 feet) from the reference element 14 (FIG. 13), a second length-indicating element 16b positioned at a second distance (4.5 feet) from the reference element (FIG. 14), a third length-indicating element 16c positioned at a third distance (5.0 feet) from the reference element (FIG. 15), a fourth length-indicating element 16d positioned at a fourth distance (5.5 feet) from the reference element (FIG. 16), a fifth length-indicating element 16e positioned at a fifth distance (6.0 feet) from the reference element (FIG. 17), a sixth length-indicating element 16f positioned at a sixth distance (6.5 feet) from the reference element (FIG. 18), and a seven length-indicating element 16g positioned at a seventh distance (7.0 feet) from the reference element (FIG. 19). (These length-indicating elements 16a-g are collectively referred to herein as the length-indicating elements 16.) For embodiments for use for adult and pediatric patients, one of the length-indicating elements (e.g., the third length-indicating element 16c of the depicted embodiment) can divide the length-indicating segments of the tape body 12 into an adult portion and a pediatric portion and can include a marking or display clearly indicating this division.

In other embodiments, more or fewer of the length-indicating elements 16 are provided, for example in adult-only designs (e.g., with a first of the length-indicating elements 16 being positioned 5.0 feet from the reference element 14) or pediatric-only designs (e.g., with a last of the length-indicating elements 16 being positioned 5.0 feet from the reference element). Also, while the depicted embodiment includes the length-indicating elements 16 in half-foot increments, in other embodiments the increments can be longer (e.g., one-foot increments) or shorter (e.g., one-inch or two-inch increments), as may be desired.

Furthermore, the area on the tape body 12 between adjacent ones of the length-indicating elements 16 defines a segment or panel 19 corresponding to one of the adjacent length-indicating elements (e.g., the lower value/height, as depicted). For example, the depicted embodiment includes a first length-indicating segment 19a spanning a first distance range (3.5 feet to 4.0 feet) from the reference element 14 (FIG. 13), a second length-indicating segment 19b spanning a second distance range (4.0 feet to 4.5 feet) from the reference element (FIG. 14), a third length-indicating segment 19c spanning a third distance range (4.5 feet to 5.0 feet) from the reference element (FIG. 15), a fourth length-indicating segment 19d spanning a fourth distance range (5.0 feet to 5.5 feet) from the reference element (FIG. 16), a fifth length-indicating segment 19e spanning a fifth distance range (5.5 feet to 6.0 feet) from the reference element (FIG. 17), a sixth length-indicating segment 19f spanning a sixth distance range (6.0 feet to 6.5 feet) from the reference element (FIG. 18), and a seven length-indicating segment 19g spanning a seventh distance range (6.5 feet to 7.0 feet) from the reference element (FIG. 19). (These length-indicating segments 19a-g are collectively referred to herein as the length-indicating segments 19.) This helps with identifying the patient's height if the patient's height is between the increments and the patient is lying on and covering (including at least partially obscuring from view) the corresponding length-indicating element 16. And to further enable ease of use, these corresponding length-indicating segments 19 can be color-coded to the length-indicating elements 16, for example the two tape segments 19c-d between 5.0 feet and 6.0 feet (e.g., FIGS. 15-16) can be colored red to correlate with an esophageal airway device known as the King Airway, and the two tape segments 19e-f between 6.0 feet and 7.0 feet (e.g., FIGS. 17-18) can be colored purple to correlate with the King Airway device.

Furthermore, indicia 20 corresponding to the distances/measured heights can be marked on the tape body 12 at a location adjacent to each of the corresponding length-indicating elements 16, at a location within the corresponding length-indicating segment 19 but distal from the corresponding length-indicating element (e.g., adjacent the next-higher length-indicating element, as depicted), or both with at least two of the indicia for each length-indicating element (as depicted). For example, the indicia 20 can be made by printing, embossing, etching, or other conventional marking techniques. The indicia 20 include literal expressions of the corresponding length-indicating elements 16 (e.g., numerals and units) and thus enable the user to conduct a quick visual check confirming the indicated indicia against a quick visual estimation of the patient's height for any obvious errors in the event the tape 10 has slipped. For example, the depicted embodiment includes first indicia 20a (“4 FT”) positioned adjacent the corresponding length-indicating element 16a and also positioned within the corresponding length-indicating segment 19a (but distal from the corresponding length-indicating element), second indicia 20b (“4.5 FT”) positioned adjacent the corresponding length-indicating element 16b and also positioned within the corresponding length-indicating segment 19b (but distal from the corresponding length-indicating element), third indicia 20c (“5 FT”) positioned adjacent the corresponding length-indicating element 16c and also positioned within the corresponding length-indicating segment 19c (but distal from the corresponding length-indicating element), fourth indicia 20d (“5.5 FT”) positioned adjacent the corresponding length-indicating element 16d and also positioned within the corresponding length-indicating segment 19d (but distal from the corresponding length-indicating element), fifth indicia 20e (“6 FT”) positioned adjacent the corresponding length-indicating element 16e and also positioned within the corresponding length-indicating segment 19e (but distal from the corresponding length-indicating element), sixth indicia 20f (“6.5 FT”) positioned adjacent the corresponding length-indicating element 16f and also positioned within the corresponding length-indicating segment 19f (but distal from the corresponding length-indicating element), and seventh indicia 20g (“7 FT”) positioned adjacent the corresponding length-indicating element 16g and also positioned within the corresponding length-indicating segment 19g (but distal from the corresponding length-indicating element). (These indicia 20a-g are collectively referred to herein as the indicia 20.)

In other embodiments, the length-indicating elements include and are defined at least in part by such indicia and/or such segments, in addition to or instead of scale lines or the like. And in still other embodiments, the length-indicating elements and the ventilator-setting elements are combined into single elements for example with the ventilator-setting elements positioned on the tape body at locations that correspond to the correlated length/height of the patient in order to thus also function as the length-indicating elements.

The ventilator-setting elements 18 listed on the tape body 12 are ventilator settings that are widely accepted in medicine and can be readily verified through a number of websites and medical references. The ventilator-setting elements 18 are correlated to the length-indicating elements 16 and enable the user to identify a proper ventilator setting based on the aligned length-indicating element corresponding to the patient's height. The ventilator-setting elements 18 can be formed by markings on the tape body 12, as depicted in FIGS. 13-19. For example, the ventilator-setting elements 18 can be made by printing, embossing, etching, or other conventional marking techniques. The ventilator-setting elements 18 are positioned on the tape body 12 to correspond to respective length-indicating elements 16 (e.g., adjacent them), with the correlation as described below. As such, with the reference element 14 in position and the tape body 12 in an extended/elongated use position alongside the patient, the aligned length-indicating element 16 corresponding to the patient's height identifies the corresponding ventilator-setting element 18.

As mentioned, the ventilator-setting elements 18 correspond to the length-indicating elements 16. So in the depicted embodiment with seven length-indicating elements 16a-g, there are at least seven corresponding ventilator-setting elements 18a-g (collectively referred to herein as the ventilator-setting elements 18). For each length-indicating element 16, the corresponding ventilator-setting element 18 can include a plurality of ventilator settings. For example, in the depicted embodiment the pediatric ventilator-setting elements 18a-b each include lists (e.g., an array) of optional ventilator settings broken out by different patient weights (24/26/28 kg or 30/32/34 kg) and by different tidal volumes, and the adult ventilator-setting elements 18c-g each include a list of optional ventilator settings broken out by patient gender (male or female) and by tidal volumes (6 mL/kg for below-normal oxygen needs, 8 mL/kg for normal, and 10 mL/kg for above-normal needs).

The ventilator-setting elements 18 literally express the appropriate respiration tidal volumes corresponding to the ideal body weight corresponding to the patient's measured height. These tidal volumes thus represent approximations that are sufficient and close-enough for use in urgent-care situations and that are readily identifiable by using the tape 10 in such urgent-care situations, not exact tidal volumes as can be more-accurately determined in non-emergency situations when time is not so critical. In other embodiments, the ventilator-setting elements are visual representations of control positions of the ventilator, for example for use with ventilators having control positions indentified by numbers, letters, icons, or the like.

These listed tidal volumes are determined by identifying the ideal weight given the patient's height and age, and then determining the appropriate tidal volume for that weight (given the patient's gender and condition, for adults), based on the following height-to-weight and weight-to-tidal-volume formulas:
Male adult weight: 50 kg (based on 60 inches, i.e., 5.0 feet)+2.3 kg for each inch over 60 inches;
Female adult weight: 45.5 kg (based on 60 inches)+2.3 kg for each inch over 60 inches;
Pediatric weight: 8+(2×age in years), known as “The Best-Guess Method” of calculating ideal weight for pediatrics 1-5 years of age and included only as a reference for the critical-care provider; and
Tidal volume: weight×tidal rate, using a weight resulting from the appropriate one of the above height-to-weight formulas, and a tidal volume of for example 8 m L/kg.
   In this way, the tape 10 is indexed for reading by a critical-care provider to quickly and easily, in a one-step read-and-set process, set up a mechanical ventilator with ventilator settings to provide appropriate tidal volumes for the patient's lungs based on the patient's height and corresponding ideal body weight.

In typical embodiments such as that depicted, the ventilator-setting elements 18 each include multiple ventilator settings, as noted above. For example, referring to FIG. 13, the first ventilator-setting element 18a can include a column including the ideal body weight (e.g., 24 kg) for a 4.0-foot tall pediatric patient as a heading and below that one or more (e.g., three) appropriate tidal volumes (e.g., 144 mL, 192 mL, and 240 mL) from which the clinician can select based on for example patient diagnosis and current hemodynamic status. Furthermore, the first ventilator-setting element 18a can include an array with additional (e.g., two) additional column headings (e.g., 26 kg and 28 kg) corresponding to ideal weights for pediatric patients with heights between the length-indicating elements, with below each heading one or more (e.g., three) corresponding appropriate tidal volumes (156 mL, 208 mL, and 260 mL, and 168 mL, 224 mL, and 280 mL).

As another example, referring to FIG. 15, the third ventilator-setting element 18c can include the ideal body weight for a 5.0 foot adult male (50.0 kg, indicated by “M”) and for a 5.0 foot adult female (45.5 kg, indicated by “F”) as a heading, and below that one or more (e.g., three M and three F) tidal volumes corresponding to one or more (e.g., three) tidal rates (e.g., at 6 mL/kg, 300 mL for male and 273 mL for female; at 8 mL/kg, 400 mL for male and 364 mL for female; and at 10 mL/kg, 500 mL for male and 455 mL for female). These listed tidal rates of 6 mL/kg, 8 mL/kg, and 10 mL/kg are the most common rates used for ventilators, with the particular tidal rate to be used selected by the clinician based on for example patient diagnosis and current hemodynamic status.

The tidal volumes of the ventilator-setting elements 18 displayed on the tape 10 as depicted are determined based on the formulas/criteria set forth above. It will be understood that additional tidal volumes of additional ventilator-setting elements for patient heights falling between the length-indicating elements 16 can be determined and included on the tape 10 based on these same formulas/criteria. In addition, it will be understood that variations of these formulas/criteria can be used, for example by using a different starting height and corresponding ideal body weight (instead of 5.0 feet and 50.0/45.5 kg M/F) and/or using average body weight for a given height (instead of ideal body weight) then making additions based on the height and/or weight of the patient as determined using the tape, or by not using a starting height and doing a calculation for the total measured height (and corresponding ideal body weight) of the patient.

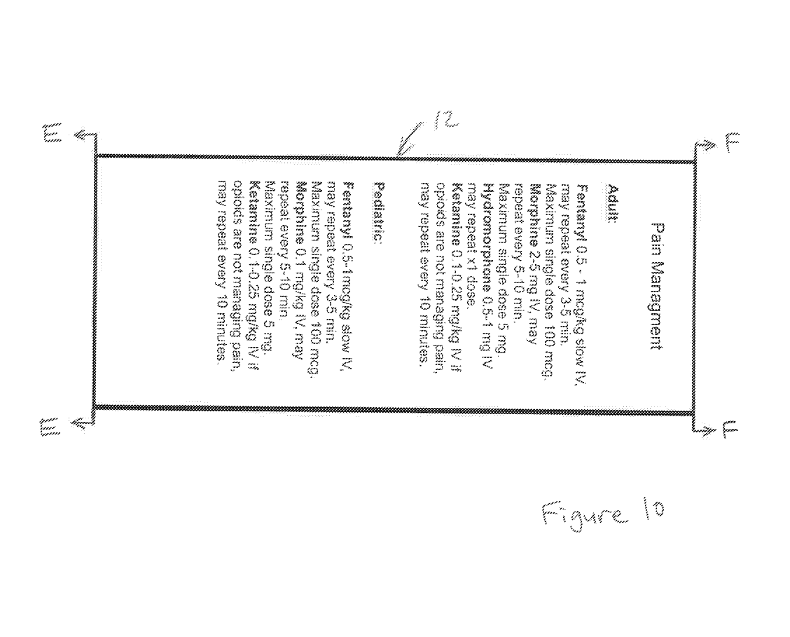

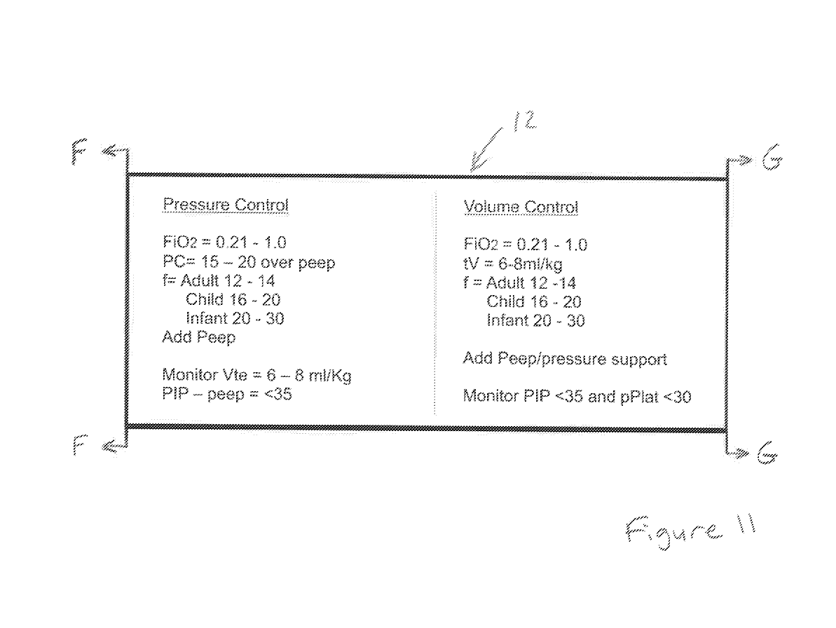

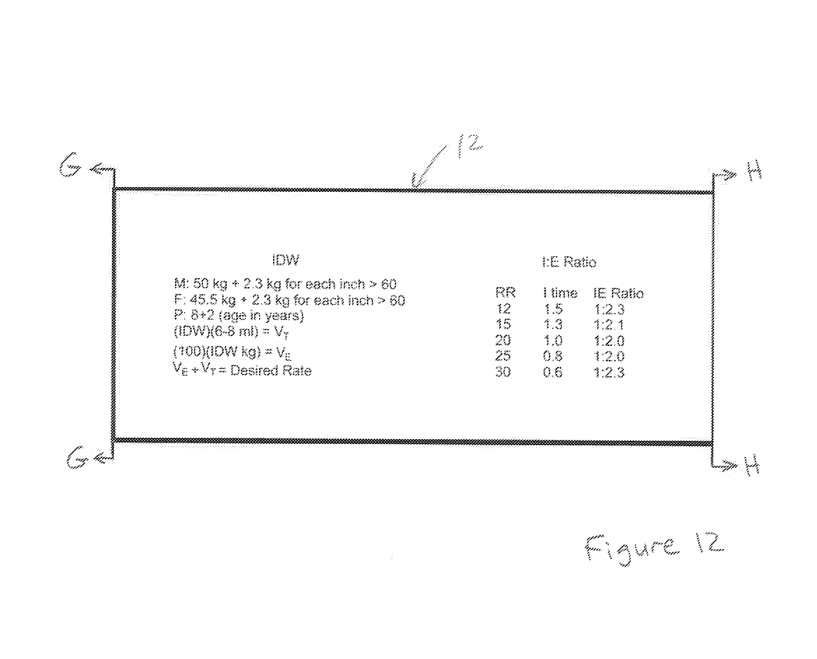

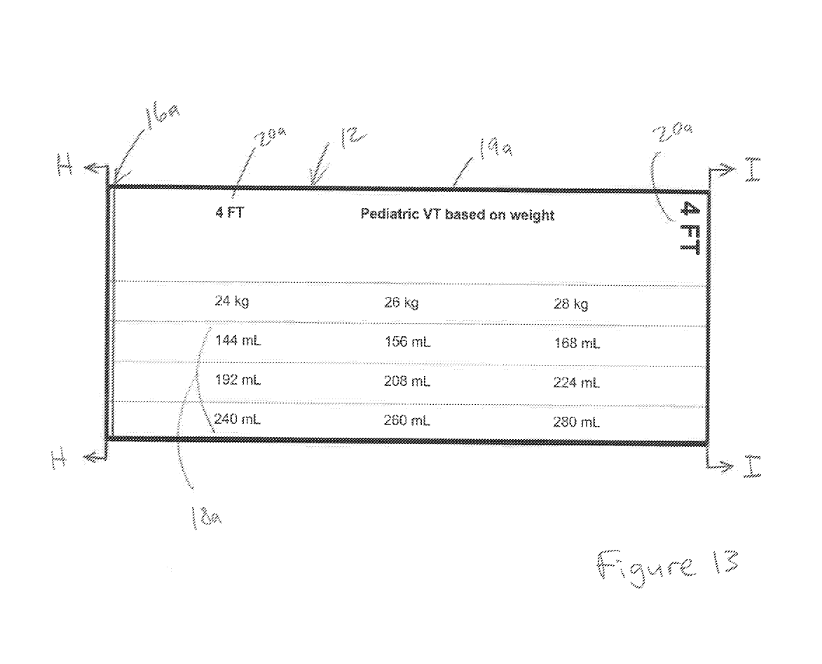

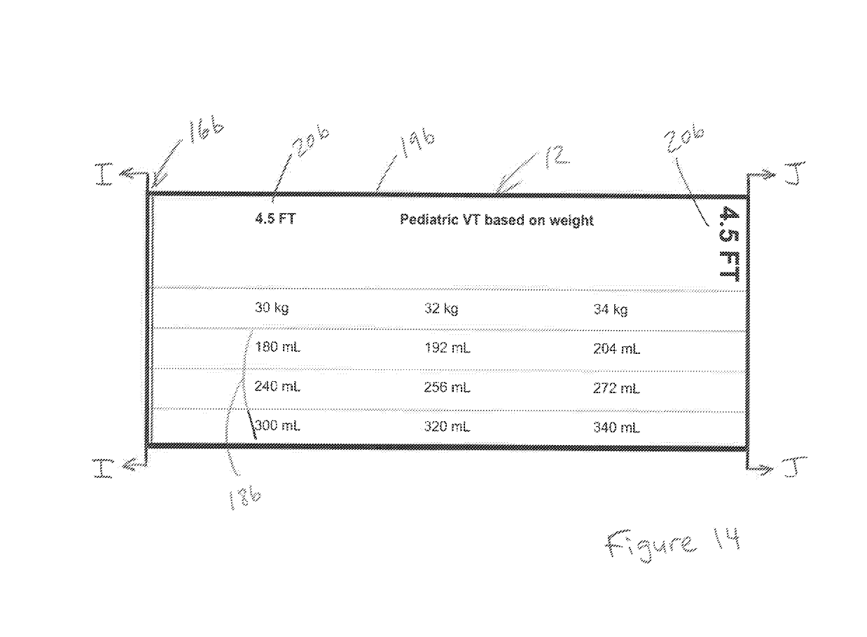

The portion of the tape 10 between the reference element 14 and the first of the length-indicating elements 16 provides the needed length and is thus included for physical extension and connection purposes. This intermediate/extension portion of the tape 10 can be significant, typically having a length that is generally comparable to the length of the length-indicating segments 19 and thus about half the overall length of the tape 10, for example with each about 3.5 feet in the depicted embodiment (in shorter pediatric-only embodiments the extension portion is typically more than half the total tape length). In addition, this intermediate/extension portion of the tape 10 can be divided into segments and used to display additional relevant information. In the depicted embodiment, for example, this intermediate/extension portion of the tape 10 is divided into serial segments that display:
Normal I:E (inhale/exhale) ratios based on respirations per minute delivered (FIG. 12);
Formulae for initial ventilator settings for a patient in volume-controlled ventilation such as the height-to-weight formulas set forth above (FIG. 12);
Initial ventilator settings for adult, child, and infant patients in pressure-control settings (FIG. 11);
Pain-management medications and doses for adult and pediatric patients (FIG. 10);
Induction doses of medication utilized to sedate a patient and paralyze a patient for rapid-sequence intubation and ventilation of adult and pediatric patients (FIG. 9);
Post-induction medications for providing sedation and comfort for ventilated adult and pediatric patients (FIG. 8); and
The Ramsay scale (listed and described), a measurement of a patient's sedation level utilized to ensure patient sedation (FIG. 7). In other embodiments, additional and/or alternative information can be displayed on this portion of the tape 10, or if desired this area can be left blank.

Referring back to FIGS. 1-4, the indexing tape 10 is shown in use in an example method of use. First, the indexing tape 10 is positioned with the reference element 14 positioned at an end of the patient, for example the sole/bottom of the patient's foot, with the patient in a supine position, for example lying down flat on a support surface, as shown. The patient's foot can be lifted slightly to slip the tape 10 under it and position the reference element 14 directly beneath the sole of that foot at the heel, then the foot can be lowered back down until its rests upon the support surface. Alternatively, the tape 10 can be positioned laterally offset slightly from the patient's foot, with the reference element 14 aligned with and beside (but not underneath) the foot.

In addition, the corresponding first end 13 of the tape body 12 can be secured in place to prevent the tape 10 from slipping when it is extended along the patient. In typical embodiments such as that depicted, the first end 13 of the tape body 12 can be secured in place by applying a strip of conventional medical adhesive tape 11 to it and to the patient's foot (or alternatively to the support surface, provided that the patient doesn't move longitudinally). In other embodiments, the reference element 14 or tape first end 13 includes a mechanical feature (e.g., an adhesive portion with a removable backing, a rigid transverse/perpendicular tab, or a transverse loop or hook) for engagement by a body part of the patient (e.g., sole or toe of foot, or crown or ear of head), and the mechanical feature is positioned in engagement with the patient to secure the tape 10 in place during use to prevent slippage and thus prevent a patient-height misreading. Furthermore, for embodiments with the reference element 14 located distally offset from the first end 13 the tape body 12 (FIGS. 1-4), the flap segment 11 that can be folded/pivoted up (e.g., to generally perpendicular or another angle relative to the tape extension segments) about the reference element 14 and onto the sole of the patient's foot and secured there for example by adhesive tape 9, as shown in FIGS. 3-4. Also, the flap segment 11 can include a blank space where the clinician can write information relating to the patient.

Then the indexing tape 10 is positioned in an extended/elongated configuration at least coextensive with the height of the patient (i.e., lying-down length). The tape 10 can be slipped entirely under the patient if the patient's condition permits, or if not then it can be extended alongside the patient with at least portions of it laterally offset from the patient as shown. With the tape 10 now extending foot-to-head along the patient, the opposite end of the patient (in this case the top of the patient's head) and a location on the tape 10 are now adjacent and in alignment (i.e., along a line perpendicular to the tape). That aligned location of the patient's head and the tape 10 is then identified by the clinician to determine the corresponding generally aligned length-indicating element 16 for the particular patient, for example the closest one of the length-indicating elements (whether in the shorter or longer direction) or the last one that has been exceeded by the patient's height.

The length-indicating elements (i.e., height markings) 16 are correlated to body weight (e.g., ideal), which are in turn correlated to appropriate ventilator-setting elements (i.e., tidal volumes) 18, which are displayed on the tape in positions corresponding to the associated length-indicating elements. So the clinician simply reads the ventilator-setting element 18 on the tape 10 that corresponds to the length-indicating element 16 (or segment 19) adjacent and aligned with the top of the patient's head to obtain an appropriate tidal volume for setting the ventilator, without having to estimate any body characteristic (e.g., height or weight) of the patient, without having to look up any conversion factors, and without having to perform any calculation.

To illustrate this one-step read-and-set method, its use will be described for a 6-foot, 1-inch male adult. With the tape 10 in its extended/elongated configuration and positioned alongside the supine patient, and with the reference element 14 at the patient's heel, the top of the patient's head would be a little beyond the fifth length-indicating element 16e. So the clinician could quickly and easily select, from the corresponding fifth ventilator-setting element 18e, a tidal volume of 465.6 mL, 620.8 mL, or 776 mL, depending on the tidal rate (6 mL/kg, 8 mL/kg, or 10 mL/kg, respectively) selected as appropriate given the patient's condition at the time. In this way, the clinician can quickly and reliably ensure that the patient receives the proper respiratory ventilation, without worry of significant overestimation or underestimation, and then move on to meeting other urgent-care needs.

This one-step read-and-set method provides for reading an appropriate tidal volume directly from the tape 10 based on the ideal body weight for the measured height of the patient, without any calculations or conversions (e.g., accounting for ideal body weight). For situations such as this example in which the patient's height is between two of the length-indicating elements 16, this provides a general approximation of appropriate tidal volumes, which is typically sufficient because the differences over only a few inches of height are not significant (i.e., the general estimation is sufficient for proper patient care).

However, if the patient's height is between length-indicating elements 16 and more precision is desired, then a more-precise tidal volume can be readily determined using the tape 10. Using this same scenario, the above-listed formulas for a male adult can be applied to a 6-foot, 1-inch height as follows: 50 kg+2.3 kg/inch (21 inches)=50 kg+48.3 kg=98.3 kg of ideal body weight (IDW). The clinician would then take that weight and multiple it by the selected oxygen tidal rate (e.g., standard/convention is 6-10 mL/kg). Therefore, this patient's more-precise initial ventilator settings for tidal volume would be 590 mL (at 6 mL/kg), 786 mL (at 8 mL/kg), or 983 mL (at 10 mL/kg).

It is to be understood that this invention is not limited to the specific devices, methods, conditions, or parameters described and/or shown herein, and that the terminology used herein is for the purpose of describing particular example embodiments by way of example only. Thus, the terminology is intended to be broadly construed and is not intended to be unnecessarily limiting of the claimed invention. For example, as used in the specification including the appended claims, the singular forms “a,” “an,” and “one” include the plural, the term “or” means “and/or,” and reference to a particular numerical value includes at least that particular value, unless the context clearly dictates otherwise. In addition, any methods described herein are not intended to be limited to the sequence of steps described but can be carried out in other sequences, unless expressly stated otherwise herein.

While the invention has been shown and described in example forms, it will be apparent to those skilled in the art that many modifications, additions, and deletions can be made therein without departing from the spirit and scope of the invention as defined by the following claims.

What is claimed is:

1. An indexing tape for determining settings for a ventilator for providing ventilation to a patient, the indexing tape comprising:
an elongate tape body having a length that is long enough to measure the height of the patient and a width configured to provide a display area, the tape body having a first end and an opposite second end defining the length;
a reference element formed on or by the tape body and configured to be positioned at a first end of the patient;
a plurality of length-indicating elements positioned serially on the tape display area, spaced at incremental distances from the reference element, corresponding to respective heights of the patient, and configured so that a generally aligned one of the length-indicating elements positioned at a second end of the patient indicates the patient's height; and
a plurality of ventilator-setting elements positioned on the tape display area and correlated to a plurality of body weights which are in turn correlated to the length-indicating elements,
wherein in use the tape body is positioned alongside the patient with the reference element at the first end of the patient and with the aligned length-indicating element at the second end of the patient, wherein the respective ventilator-setting element corresponding to the aligned length-indicating element represents an appropriate setting for the ventilator.

2. The indexing tape of claim 1, wherein the ventilator-setting elements include literal expressions of respiratory tidal volumes.

3. The indexing tape of claim 1, wherein the tape-body display area includes a plurality of length-indicating segments that are arranged serially and that are each defined by and between two sequential ones of the serial length-indicating elements.

4. The indexing tape of claim 3, wherein the ventilator-setting elements are positioned and displayed on corresponding ones of the length-indicating segments.

5. The indexing tape of claim 3, wherein at least one of the length-indicating segments is color coded.

6. The indexing tape of claim 3, wherein the tape body includes an extension portion between the reference element and the length-indicating segments.

7. The indexing tape of claim 6, wherein the extension portion has a length that is about half or more of the overall length of the tape.

8. The indexing tape of claim 1, wherein at least one of the length-indicating elements and at least a corresponding one of the ventilator-setting elements is for use for pediatric patients, and at least another one of the length-indicating elements and at least a corresponding other one of the ventilator-setting elements is for use for adult patients.

9. The indexing tape of claim 1, wherein the tape-body display area includes a plurality of indicia each corresponding to a respective one of the length-indicating elements and each including a literal expression of the corresponding incremental distance from the reference element to provide a check against a visual estimation of the patient's height.

10. The indexing tape of claim 1, wherein the reference element is positioned distally offset from the first end of the tape body, wherein a flap segment of the tape body is formed between the reference element and the tape-body first end, and wherein the tape body can be folded at the reference element to pivot the flap segment to a position angled relative to segments of the tape body having the length-indicating elements.

11. The indexing tape of claim 10, wherein in the angled position, the flap segment can be secured to the patient to prevent tape slippage during use and thus prevent a misreading.

12. A method of providing ventilation to a patient, the method comprising:
positioning an elongate indexing tape in an extended/use configuration along the patient with a reference element of the indexing tape at a first end of the patient;
extending the indexing tape until a second opposite end of the patient is generally aligned with one of a plurality of length-indicating elements of the indexing tape that are serially arranged and spaced incremental distances from the reference element to correspond to patient heights, which are in turn correlated to a plurality of patient body weights, which are in turn correlated to a plurality of ventilator-setting elements including oxygen tidal volumes positioned on the indexing tape;
identifying one of the length-indicating elements that generally aligns with the patient second end;
reading one of the ventilator-setting elements that corresponds to the aligned length-indicating element to identify the appropriate respiratory tidal volume for the patient; and
setting a mechanical ventilator using the read ventilator-setting element corresponding to the aligned length-indicating element to provide appropriate ventilation for the patient.

13. The method of claim 12, wherein all the steps are performed to identify the appropriate respiratory tidal volume for the patient without estimating any body characteristic of the patient, without looking up any conversion factors, and without performing any calculation.

14. The method of claim 12, wherein the step of reading one of the ventilator-setting elements includes reading one of the ventilator-setting elements from one of a plurality of length-indicating segments that are arranged serially and that are each defined by two sequential ones of the serial length-indicating elements.

15. The method of claim 12, wherein the step of reading one of the ventilator-setting elements includes selecting one of the respiratory tidal volumes corresponding to the aligned length-indicating element based on the condition of the patient.

16. The method of claim 12, further comprising the step of conducting a check against a visual estimation of the patient's height by reading an indicia on the tape that corresponds to and is a literal expression of the incremental distance of the aligned length-indicating element from the reference element.

17. The method of claim 12, further comprising the step of folding up a flap segment of the tape body, at the reference element, to an angled position against the first end of the patient, wherein the reference element is positioned distally offset from the first end of the tape body to form the flap segment therebetween.

18. The method of claim 17, further comprising the step of securing the flap segment of the tape body against the first end of the patient to prevent tape slippage during the step of extending the indexing tape.

19. The method of claim 18, wherein the step of securing the flap segment includes applying a strip of adhesive tape to the flap segment and the patient.

20. The method of claim 12, wherein the step of identifying one of the length-indicating elements that generally aligns with the patient second end includes identifying the closest one of the length-indicating elements to the patient second end or identifying the last one of the length-indicating elements that has been exceeded by the patient's height.
